(19) United States
(12) Patent Application Publication
(10) Pub. No.: US 2024/0110863 A1
(43) Pub. Date: Apr. 4, 2024

(54) INFORMATION PROCESSING DEVICE, INFORMATION PROCESSING METHOD, AND PROGRAM

(71) Applicant: Sony Group Corporation, Tokyo (JP)

(72) Inventor: Tuo ZHUANG, Tokyo (JP)

(73) Assignee: Sony Group Corporation, Tokyo (JP)

(21) Appl. No.: 18/276,597

(22) PCT Filed: Mar. 1, 2022
(86) PCT No.: PCT/JP2022/008688

§ 371 Date: Aug. 9, 2023

(30) Foreign Application Priority Data
Mar. 15, 2021 (JP) 2021-041185

(51) Int. Cl. G01N 21/31; G01N 33/02

(52) U.S. Cl. CPC G01N 21/31; G01N 33/02; G01N 2201/0635

(57) ABSTRACT

An information processing device according to an embodiment includes: a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other; a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations; and a generation unit that generates a point spread function of each wavelength based on the third combination of each pixel specified by the specifying unit.

### FIELD

The present disclosure relates to an information processing device, an information processing method, and a program.

### BACKGROUND

Conventionally, a spectroscopic measurement method is known as an object composition analysis method. The spectroscopic measurement method is a method for analyzing radiation light, reflection light, or transmission light from an object, and thereby analyzing a composition (elements, molecular structures, and the like) of the object.

A light wavelength component of radiation light, reflection light, or transmission light from an object varies depending on a composition of the object. Therefore, it is possible to analyze the composition of the object by analyzing this wavelength component of the radiation light, the reflection light, or the transmission light. In general, data indicating a quantity of each wavelength is referred to as a wavelength spectrum, and processing of measuring a wavelength spectrum is referred to as spectroscopic measurement processing.

To analyze the composition at each point on a surface of the object, it is necessary to acquire corresponding data of spatial information and wavelength information of the object. As a method for acquiring the corresponding data of the spatial information and the wavelength information of the object by processing the corresponding data of the spatial information and the wavelength information of the object once, that is, only by performing photographing processing of the spectroscopic measurement device once, a snapshot system is known. A spectroscopic measurement device to which the snapshot system is applied includes a combination of an optical system including a plurality of lenses, slits (field diaphragm), spectral elements, and the like, and a sensor. Spatial resolution and wavelength resolution of the spectroscopic measurement device are determined according to configurations of these optical systems and sensor.

### CITATION LIST

### Patent Literature

Patent Literature 1: JP 2016-90576 A

### SUMMARY

### Technical Problem

Here, although the spectroscopic measurement device needs to acquire a Point Spread Function (PSF) of each spatial position and each wavelength by calibration to perform restoration processing on a captured image, there is a problem that a conventional calibration method takes a longer time spent for calibration as a necessary visual field range and wavelength range are widened more, and requires more cost.

Therefore, the present disclosure proposes an information processing device, an information processing method, and a program that can more easily acquire a PSF.

### Solution to Problem

To solve the problems described above, an information processing device according to an embodiment of the present disclosure includes: a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other; a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations; and a generation unit that generates a point spread function of each wavelength based on the third combination of each pixel specified by the specifying unit.

### BRIEF DESCRIPTION OF DRAWINGS

FIG. 1 is a view for describing a relationship between a type and a wavelength of light.

FIG. 2 is a view for describing an example of spectroscopic measurement of a light emitting object.

FIG. 3 is a view illustrating an example of a spectral intensity analysis result that is a spectroscopic analysis result of output light of certain food.

FIG. 4 is a view for describing a prism that is a spectral element.

FIG. 5 is a view for describing an explanation grating that is a spectral element.

FIG. 6 is a view for describing an example of a data cube that is data having three dimensions of a spatial direction (XY) and a wavelength direction (λ) of a measurement target.

FIG. 7 is a view illustrating a schematic configuration example of a spectroscopic measurement device of a snapshot system.

FIG. 8 is a view illustrating an example of data acquired by performing photographing processing once using the spectroscopic measurement device of the snapshot system.

FIG. 9 is a view for describing a captured image photographed by the spectroscopic measurement device of the snapshot system according to an embodiment of the present disclosure.

FIG. 10A is a view for describing a luminance value of each pixel of a diffraction image photographed by the spectroscopic measurement device according to the embodiment of the present disclosure (part 1).

FIG. 10B is a view for describing a luminance value of each pixel of a diffraction image photographed by the spectroscopic measurement device according to the embodiment of the present disclosure (part 2).

FIG. 10C is a view for describing a luminance value of each pixel of a diffraction image photographed by the spectroscopic measurement device according to the embodiment of the present disclosure (part 3).

FIG. 11 is a view for describing a restoration method for restoring a data cube from a diffraction image acquired by the spectroscopic measurement device according to the embodiment of the present disclosure.

FIG. 12 is a view illustrating an example of a device configuration for executing calibration for acquiring a point spread function of each spatial position and each wavelength.

FIG. 13 is a view for describing an example of a flow of calibration that uses the device configuration illustrated in FIG. 12.

FIG. 14 is a view for describing another example of the flow of calibration that uses the device configuration illustrated in FIG. 12.

FIG. 15 is a view for describing a mechanism that diffraction patterns of respective wavelengths are superimposed on a diffraction image according to the embodiment of the present disclosure.

FIG. 16 is a flowchart of an operation example according to the embodiment of the present disclosure.

FIG. 17 is a flowchart illustrating an operation example according to a modification of the embodiment of the present disclosure.

FIG. 18 is a view for describing a PSF restoration operation that uses a reference table according to the embodiment of the present disclosure.

FIG. 19 is a hardware configuration diagram illustrating an example of an information processing device that executes various processing according to the present disclosure.

### DESCRIPTION OF EMBODIMENTS

Hereinafter, an embodiment of the present disclosure will be described in detail with reference to the drawings. Note that, in the following embodiment, the same components will be assigned the same reference numerals, and redundant description will be omitted.

Furthermore, the present disclosure will be described in order of items described below.
1. Embodiment
1.1 Outline of Spectroscopic Measurement Device (System)
1.2 Mechanism That Diffraction Patterns are Superimposed
1.3 Condition for Making It Possible to Separate Wavelength Feature Amount
1.4 Method for Determining Wavelength Feature Amount
1.5 Example of Wavelength Feature Amount Determining Operation
1.6 Modification of Wavelength Feature Amount Determining Operation
1.7 Regarding Restoration of PSF That Uses Reference Table
1.8 Conclusion
2. Hardware Configuration

### 1. Embodiment

Hereinafter, an information processing device, an information processing method, and a program according to the present embodiment will be described in detail with reference to the drawings.

1.1 Outline of Spectroscopic Measurement Device (System)

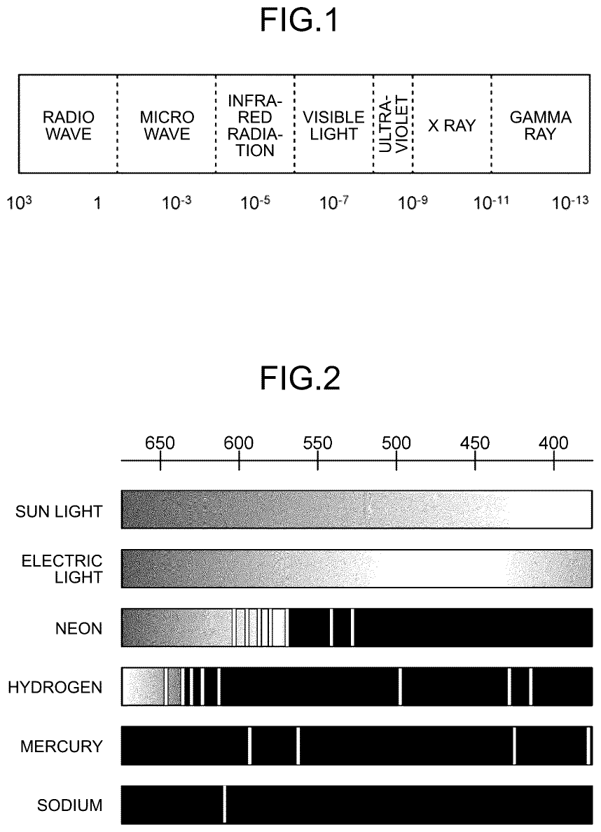

First, an outline of a spectroscopic measurement device (system) according to the present embodiment will be described. Although, for example, infrared radiation, visible light, ultraviolet, and the like are known as light, these beams of light are a kind of electromagnetic waves, and have different wavelengths (vibration cycles) depending on the type of light as illustrated in FIG. 1.

There are characteristics that the wavelength of visible light ranges from approximately 400 nm to 700 nm, and, while infrared radiation has a longer wavelength than that of visible light, ultraviolet has a shorter wavelength than that of the visible light.

As described above, a light wavelength component of radiation light, reflection light, or transmission light from an object differs depending on a composition (elements, molecular structures, and the like) of the object, and the composition of the object can be analyzed by analyzing this wavelength component. In general, data indicating a quantity of each wavelength is referred to as a wavelength spectrum, and processing of measuring a wavelength spectrum is referred to as spectroscopic measurement processing.

FIG. 2 is a view illustrating an example of spectroscopic measurement of a light emitting object. FIG. 2 illustrates light of which wavelength in a wavelength range of visible light (approximately 400 nm to 700 nm) light output from the sun, an electric light, neon, hydrogen, mercury, or sodium is. Ranges outputting light are displayed in white, and ranges outputting no light are illustrated in black. FIG. 2 illustrates results obtained by performing spectroscopic measurement on output light from sunlight, electric lights, and various heated substances.

As illustrated in FIG. 2, the sun, the electric light, the neon, the hydrogen, the mercury, the sodium, and each of these objects output wavelength light unique to each object. That is, even if the object is unknown, it is possible to analyze the composition of this object by analyzing the wavelength component contained in light from this object.

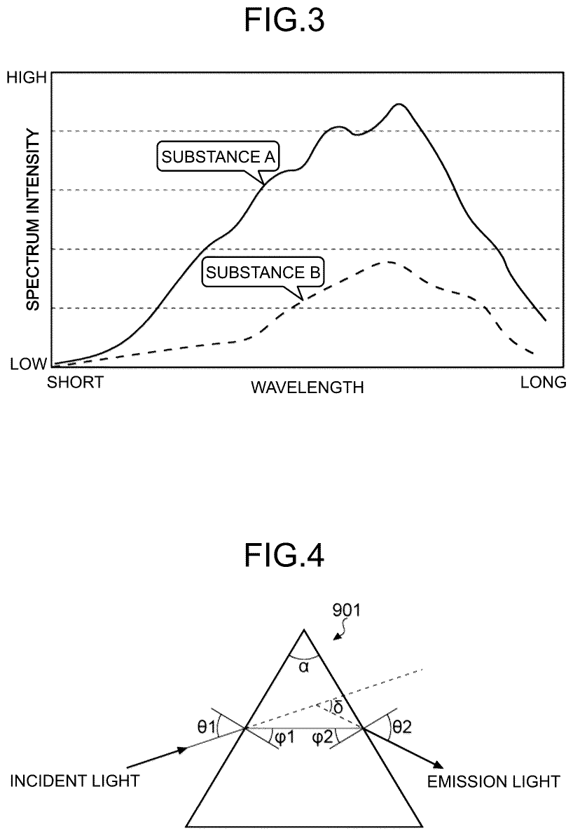

In a case where, for example, the composition of certain processed food is unknown, it is possible to analyze a substance constituting this food by analyzing output light (radiation light, reflection light, or transmission light) of this food. FIG. 3 is a view illustrating an example of a spectral intensity analysis result that is a spectroscopic analysis result of output light of certain food. Two different types of spectroscopic analysis results are obtained from this food.

By comparing this spectral intensity analysis result, and spectral intensity analysis result data of various substances analyzed in advance, it is possible to determine what a substance A and a substance B are, and it is possible to analyze the composition of food.

As described above, when spectroscopic measurement can be performed, it is possible to acquire various pieces of information related to a measurement target. However, light having all wavelengths in a mixed manner is incident on each pixel of a sensor, and therefore a general camera including a condenser lens and the sensor has difficulty in analyzing the intensity in a unit of each wavelength.

Hence, an observation system of spectroscopic measurement is provided with a spectral element (spectral device) for separating light of each wavelength from light coming into the camera.

The most commonly known spectral element is a prism 901 illustrated in FIG. 4. Light incident on the prism 901, that is, light of various wavelengths included in the incident light is emitted from the prism 901 at an emission angle matching the wavelength of the incident light, an incident angle, and the shape of the prism 901. The observation system of spectroscopic measurement is provided with a spectral element such as the prism 901, and employs a configuration where a sensor can individually receive light in units of wavelengths.

Note that an equation that expresses change in a traveling direction of light caused by a prism during light dispersion that uses the prism having a refractive index n can be expressed by following equation (1).

δ=θ₁−ϕ₁+θ₂−ϕ₂=θ₁+θ₃−α (1)

Note that each parameter of above equation (1) is as follows.
α: apex angle of prism
θ₁: incident angle with respect to prism incident surface
θ₂: emission angle with respect to prism emission surface
ϕ₁: refraction angle of prism incident surface
ϕ₂: refraction angle of prism emission surface
δ: deflection angle (angle between incident light and emission light)

Here, according to the Snell's law (sin θⱼ=n sin Φⱼ), above equation (1) can be rewritten as following equation (2).

δ=θ₁+sin⁻¹(n·sin(α−ϕ₁)) (2)

Note that, in above equation (2), n represents the refractive index of the prism, and the refractive index n depends on the wavelength. Furthermore, ϕ₁ represents a refractive angle of a prism incident surface, and depends on the refractive index n of the prism and an incident angle θ₁ with respect to the prism incident surface. Therefore, the deflection angle (an angle between incident light and emission light) δ depends on the incident angle θ₁ and the wavelength.

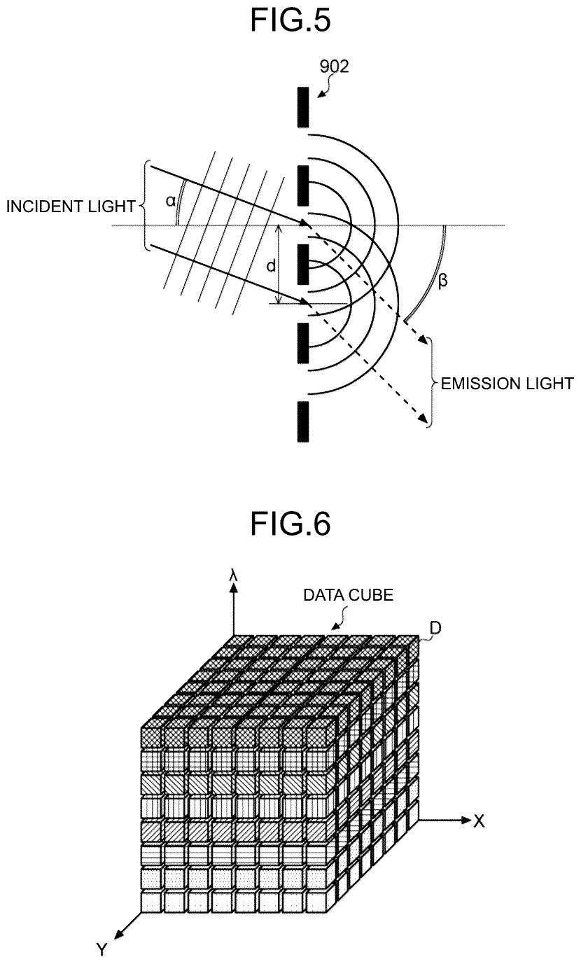

Furthermore, as illustrated in FIG. 5, a diffraction grating 902 can also perform light dispersion using the property of a wave of light. An emission angle (diffraction angle) β of a beam from the diffraction grating 902 can be expressed by following equation (3).

$$β = {sin}^{- 1} ( \frac{m \cdot λ}{d} - sin α ) \qquad ( 3 )$$

Note that, in above equation (3), d represents a grating interval, α represents an incident angle, β represents an emission angle, and m represents a diffraction order.

However, even if wavelength information of light from certain one point of an object is analyzed, the composition at this certain one point can only be analyzed. That is, to analyze the composition at each point on the surface of the object by performing observation once, it is necessary to analyze all beams of light from each point of the surface.

To analyze the composition at each point of a surface of a measurement target, it is necessary to acquire data having three dimensions of a spatial direction (XY) and a wavelength direction (λ) of the measurement target by performing observation once. FIG. 6 illustrates an example of data, that is, a data cube having three dimensions of the spatial direction (XY) and the wavelength direction (λ) of the measurement target.

As illustrated in FIG. 6, the data cube is data that has the three dimensions of the spatial direction (XY) and the wavelength direction (λ) of the measurement target, and is data that indicates coordinates at each point on the surface of the measurement target as XY coordinates, and records an intensity (λ) of each wavelength light at each coordinate position (x, y). The data cube illustrated in FIG. 6 includes cubic data of 8×8×8, and one cube D is data indicating the light intensity of a specific wavelength (λ) at a specific position (x, y).

Note that the number of cubes 8×8×8 illustrated in FIG. 6 is an example, and this number varies depending on spatial resolution and wavelength resolution of the spectroscopic measurement device.

Next, examples of existing spectroscopic measurement devices that acquire a data cube as illustrated in FIG. 6, that is, data having the three dimensions of the spatial direction (XY) and the wavelength direction (λ) of a measurement target will be described.

The existing spectroscopic measurement devices that acquire three-dimensional data of the spatial direction (XY) and the wavelength direction (λ) of the measurement target are classified into following four types.
(a) Point measurement system (spectrometer)
(b) Wavelength scan system
(c) Spatial scan system
(d) Snapshot system

Hereinafter, (d) the snapshot system among these systems will be described citing an example.

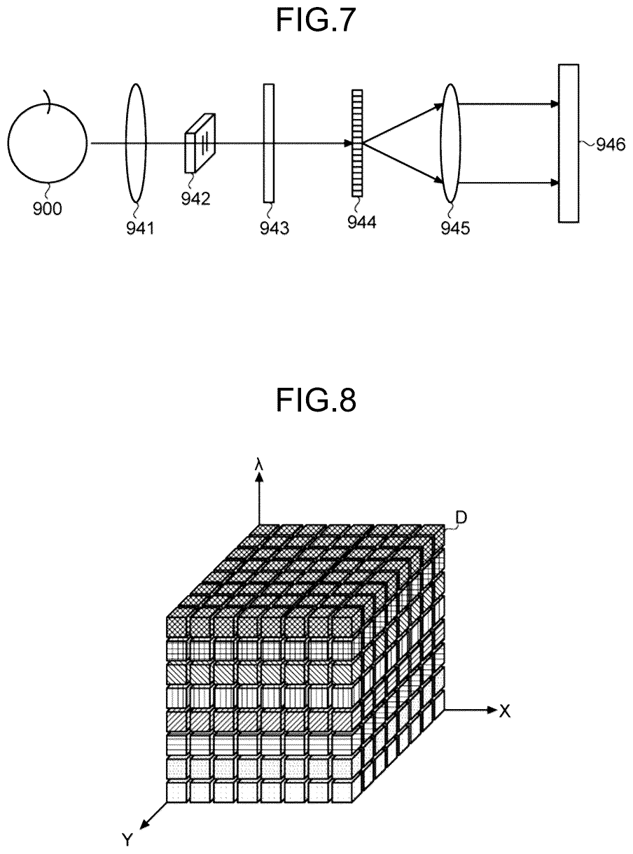

FIG. 7 is a view illustrating a schematic configuration example of the spectroscopic measurement device of the snapshot system, and FIG. 8 is a view illustrating an example of data acquired by performing photographing processing once using the spectroscopic measurement device of the snapshot system.

As illustrated in FIG. 7, the snapshot system includes an objective lens 941, a slit 942, a collimating lens 943, a diffraction grating type spectral element (hereinafter, simply referred to as a diffraction grating) 944, an imaging lens 945, and an area sensor 946, and is configured such that the objective lens 941 condenses light from a measurement target 900, and the collimating lens 943 further converts the light into parallel light to cause the light to transmit through the diffraction grating 944, and to be projected on a light reception surface of the area sensor 946. Note that the light reception surface may be a surface on which photoelectric conversion units such as photodiodes in an image sensor (also referred to as a solid-state imaging device) are aligned.

According to this configuration, light of different wavelength components from different points on the measurement target 900 is recorded in different elements (pixels) on the light reception surface of the area sensor 946.

According to this snapshot system, it is possible to acquire the data cube described with reference to FIG. 6, that is, the data cube having the three dimensions of the spatial direction (XY) and the wavelength direction (λ) of the measurement target 900 as illustrated in FIG. 8 by performing photographing once. Therefore, the snapshot system has a feature that a utility value of the acquired information is higher than those of other systems.

In this regard, a light reception area of the area sensor 946 is finite, information of the wavelength direction overlaps on the light reception surface and is recorded, and therefore it is necessary to perform processing of restoring the data cube by performing signal processing after photographing.

Furthermore, various coefficients used for the signal processing are linked with performance of an optical system, and therefore it is necessary to fix the optical system, that is, to fix the positional relationship between the sensor and the optical system to use, and there is a problem that it is difficult to adjust the wavelength and spatial resolution according to application purposes.

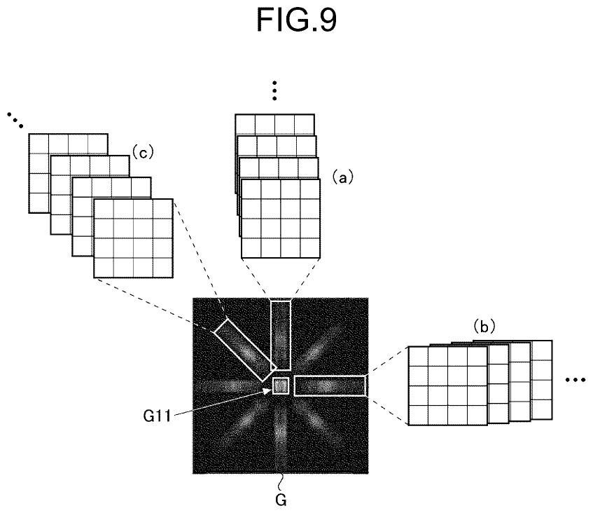

FIG. 9 is a view for describing a captured image (hereinafter, also referred to as a diffraction image) photographed by the spectroscopic measurement device of the snapshot system (hereinafter, also referred to as a spectral camera or an imaging unit). As illustrated in FIG. 9, in the spectral camera, the spectral element (e.g., diffraction grating 944) forms a beam of each wavelength at a different position on a light reception surface of a light receiving element (e.g., area sensor 946). More specifically, the beam of each wavelength is formed at a position apart from a center 0th-order diffraction pattern G11 as the wavelength becomes longer. Therefore, in a diffraction image G acquired by the spectral camera including a lattice-shaped spectral element, a diffraction pattern of each wavelength spreads in each of an upper/lower direction, a left/right direction, and four oblique directions. At that time, the diffraction pattern of each wavelength may be partially superimposed on each other. The degree of superimposition depends on the characteristics of the spectral element and a lens system. Note that, in FIG. 9, (a) illustrates an example of overlap of diffraction patterns diffracted in an upper direction from the 0th-order diffraction pattern G11, (b) illustrates an example of overlap of diffraction patterns diffracted in a right direction, and (c) illustrates an example of overlap of diffraction patterns diffracted in an oblique upper left direction.

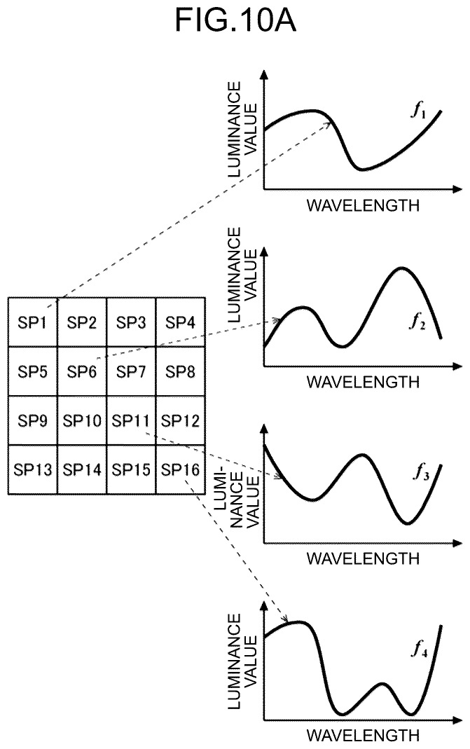

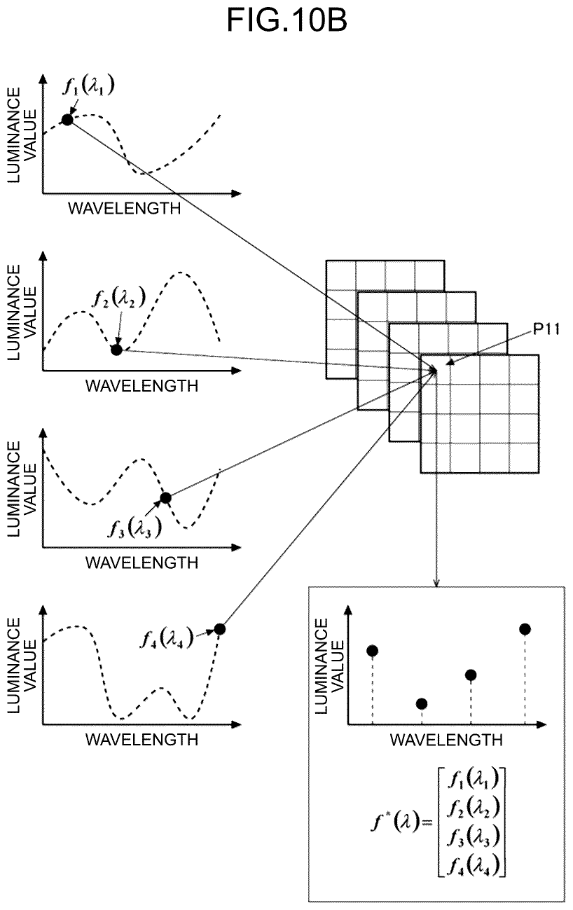

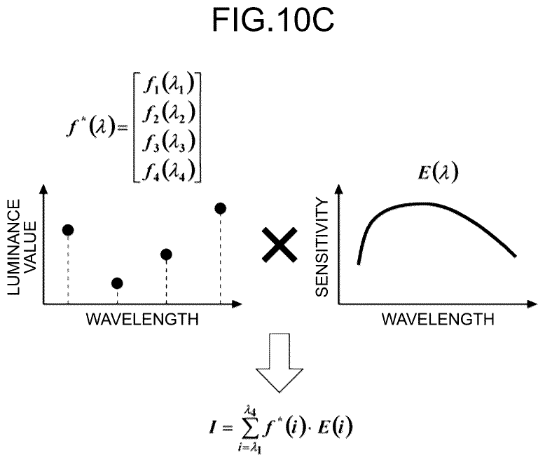

FIGS. 10A to 10C are diagrams for describing a luminance value of each pixel of a diffraction image photographed by a spectral camera, FIG. 10A is a view illustrating a wavelength spectrum of light radiated from each of spatial positions (respectively corresponding to light sources) partitioned into 4×4, FIG. 10B is a view for describing a feature amount f*(λ) of light (hereinafter, also referred to as synthesized diffracted light) obtained by synthesizing one or more beams of diffracted light incident on a specific pixel P11 located in the oblique upper left direction (corresponding to a direction in which diffraction patterns illustrated in (c) of FIG. 9 are formed) from a pixel region (a region in which the 0th-order diffraction pattern G11 is formed) on which 0th-order diffracted light is incident, and FIG. 10C is a view for describing a luminance value of synthesized diffracted light detected by the pixel P11. Note that, although FIGS. 10A to 10C illustrate cases where a 0th-order diffraction pattern and a 1st-order diffraction pattern are formed in a pixel region of 4 pixels×4 pixels that is the same as partitioning of spatial positions, and deviation of a region at which a diffraction pattern of each wavelength is formed is one pixel unit, the present invention is not limited thereto.

As illustrated in FIG. 10A, light having respectively different wavelength characteristics is emitted from each of 16 spatial positions SP1 to SP16 of 4×4 in total located at respectively spatially different locations. Here, this description focuses on the spatial positions SP1, SP6, SP11, and SP16, and assumes that light of wavelength characteristics (also referred to as spectral characteristics) f₁, f₂, f₃, and f₄ is radiated therefrom. Note that the wavelength characteristics may be, for example, characteristics including a wavelength feature amount that is a luminance value (also referred to as a light intensity) observed per wavelength from light including a plurality of wavelengths.

Here, focusing on the specific pixel P11 located in the oblique upper left direction with respect to the pixel region (the region in which the 0th-order diffraction pattern G11 is formed) on which 0th-order diffracted light is incident as illustrated in FIG. 10B, for example, a component of the wavelength λ₁ of the diffracted light (hereinafter, referred to as diffracted light of the wavelength feature amount f₁(λ₁)) of the light radiated from the spatial position SP1, a component of the wavelength λ₂ of the diffracted light (hereinafter, referred to as diffracted light of the wavelength feature amount f₂(λ₂)) of the light radiated from the spatial position SP6, a component of the wavelength λ3 of the diffracted light (hereinafter, referred to as diffracted light of the wavelength feature amount f₃(λ₃)) of the light radiated from the spatial position SP11, and a component of the wavelength λ4 of the diffracted light (hereinafter, referred to as diffracted light of the wavelength feature amount f₄(λ₄)) of the light radiated from the spatial position SP16 are incident on this pixel P11. Therefore, the feature amount (hereinafter, referred to as a synthesized feature amount) f*(λ) of the synthesized diffracted light incident on the pixel P11 can be expressed by following equation (4). Note that, in this description, the wavelength feature amount of certain light may be information for specifying light of this wavelength, and may be, more specifically, for example, a luminance value or the like of each wavelength that needs to be observed in a light receiving element (e.g., area sensor 946).

$${f}^{*} ( λ ) = [ \begin{matrix}{f}_{1} ( {λ}_{1} ) \\ {f}_{2} ( {λ}_{2} ) \\ {f}_{3} ( {λ}_{3} ) \\ {f}_{4} ( {λ}_{4} )\end{matrix} ] \qquad ( 4 )$$

Furthermore, as illustrated in FIG. 10C, a luminance value (also referred to as an observation value) I detected in the pixel P11 can be obtained by multiplying the synthesized feature amount f′(λ) with wavelength sensitivity characteristics E(λ) of the pixel P11 as expressed by following equation (5).

$$I = {\sum }_{i = {λ}_{1}}^{{λ}_{4}} {f}^{*} ( i ) \cdot E ( i ) \qquad ( 5 )$$

As described above, in the diffraction image G photographed by the spectral camera, the luminance value I of each pixel can be obtained by summing values obtained by multiplying with the wavelength characteristic E(λ) of the light receiving element for light of each wavelength the wavelength feature amount f(λ) of the diffracted light radiated from each of the observation target spatial positions SP1 to SP16 and incident on each pixel.

Next, a restoration method for restoring a data cube (see, for example, FIG. 8) from the diffraction image acquired as described above will be described. Note that, in the following description, for simplicity of description, the luminance value I of each pixel takes a value obtained by summing the wavelength feature amounts f(λ) of the diffracted light incident on each pixel, that is, a value obtained by omitting multiplication of the wavelength sensitivity characteristics E(λ) of the light receiving element.

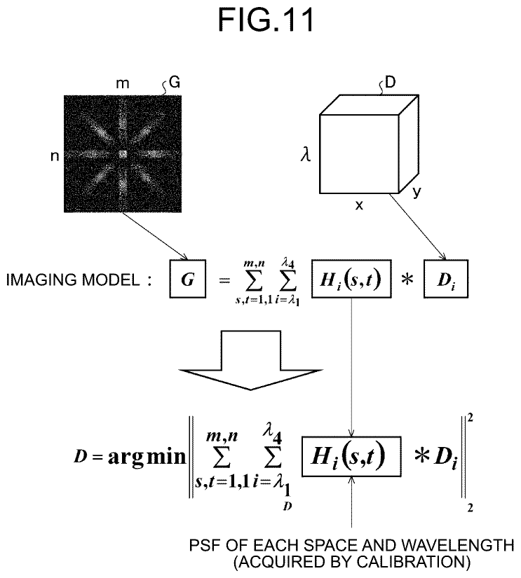

FIG. 11 is a view for describing a restoration method for restoring a data cube from a diffraction image acquired by the spectral camera. As illustrated in FIG. 11, the diffraction image G of X pixels×Y pixels (X and Y are integers equal to or more than one) acquired by the spectral camera can be modeled as in following equation (6).

$$G = {\sum }_{s , t = 1 , 1}^{X , Y} {\sum }_{i = {λ}_{1}}^{{λ}_{4}} {H}_{i} 〈 s , t 〉 * {D}_{i} \qquad ( 6 )$$

In equation (6), D₁ on the right side represents a data cube of each of wavelengths λ₁ to λ₄, and H₁ (s, t) represents a Point Spread Function (Point Spread Function: PSF) of each spatial position and each wavelength. Consequently, by solving following equation (7), it is possible to restore the data cube D in a case where the wavelengths λ₁ to λ₄ are light sources.

$$D = argmin {‖ {\sum }_{s , t = 1 , 1}^{X , Y} {{\sum }_{i = {λ}_{1}}^{{λ}_{4}}}_{D} {H}_{i} ( s , t ) * {D}_{i} ‖}_{2}^{2} \qquad ( 7 )$$

Thus, the diffraction image G acquired by the spectral camera of the snapshot system can be expressed by a convolution sum of the PSF of each spatial position and each wavelength, and the data cube Dᵢ of each wavelength. Note that, in equation (7) and other equations, (*) represents convolution. Therefore, if the PSF can be created in advance, it is possible to restore the data cube D of the diffraction image G by executing an appropriate optimization operation.

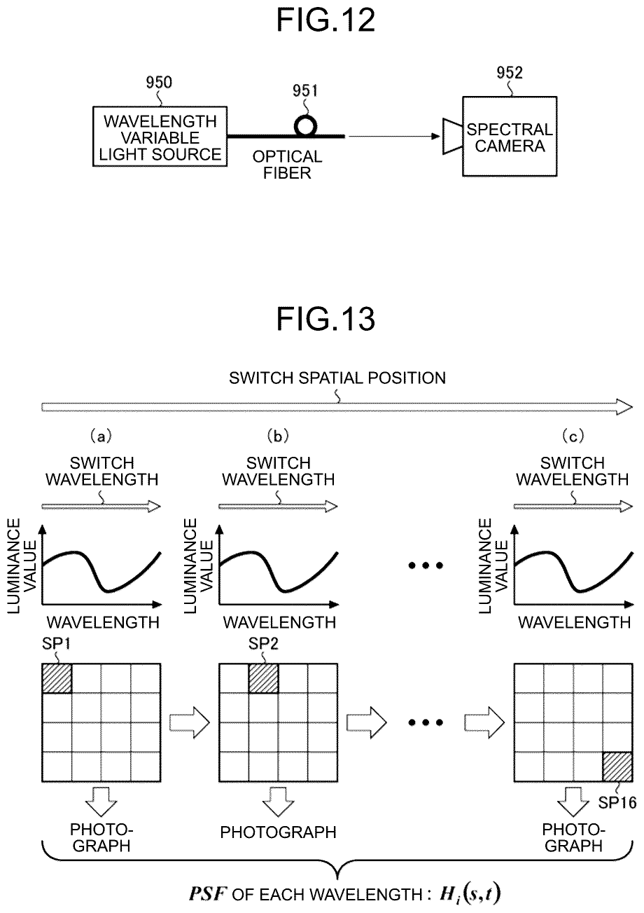

It is necessary to acquire the PSF of each spatial position and each wavelength by calibration to create Hᵢ(s, t). FIG. 12 is a view illustrating an example of a device configuration for executing calibration for acquiring the PSF of each spatial position and each wavelength, and FIG. 13 is a view for describing an example of a flow of calibration that uses the device configuration illustrated in FIG. 12.

As illustrated in FIG. 12, for example, a wavelength variable light source 950 that can adjust a wavelength is used for calibration. When light emitted from this light source 950 is incident on an optical fiber 951, a light flux width thereof is narrowed, and then the light is incident on a spectral camera 952.

According to the calibration that uses such a device configuration, first, as illustrated in (a) of FIG. 13, the spectral camera 952 executes photographing while sequentially switching the wavelength of light emitted from the wavelength variable light source 950 in a state where an emission end of the optical fiber 951 is fixed at a predetermined spatial position (the spatial position SP1 in this example). When photographing for the necessary wavelength is completed at this spatial position SP1, the position of the emission end of the optical fiber 951 is then switched to the spatial position SP2 as illustrated in (b), and, similarly, the spectral camera 952 executes photographing while sequentially switching the wavelength of the light emitted from the wavelength variable light source 950. Thus, by photographing all of the spatial positions SP1 to SP16 while switching the wavelength, the PSF of each spatial position and each wavelength is acquired.

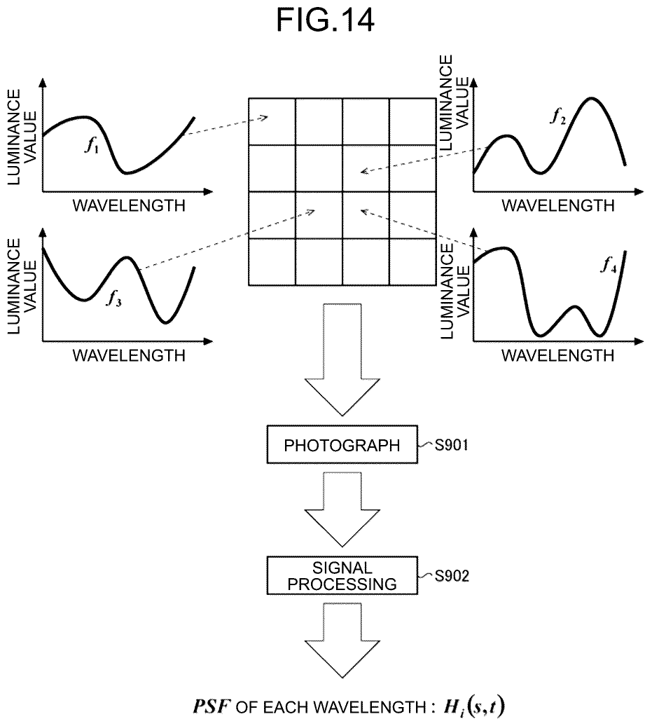

However, the above-described calibration method takes a longer time spent for calibration as a necessary visual field range and wavelength range are widened more, and requires more cost. Although, as a method for solving such a problem, there may be conceived, for example, a method for executing imaging S901 in a state where all of spatial positions (e.g., SP1 to SP16 in FIG. 10A) emitting light of respectively different wavelength spectra are simultaneously turned on as illustrated in FIG. 14, executing signal processing S902 on a diffraction image obtained by imaging S901, and thereby acquiring PSFs for all of the wavelengths by performing photographing once, such a solution method mixes different wavelength components of light of different wavelength characteristics in an observed diffraction image, and therefore has difficulty in separating the wavelength components.

Therefore, according to the present embodiment, for the purpose of solving these problems, a plurality of light emitting units (corresponding to, for example, the spatial positions SP1 to SP16) that emit light having mutually unique wavelength feature amounts are determined to make it possible to easily separate the wavelength feature amount in subsequent signal processing. Next, a combination of wavelength feature amounts for uniquely determining of which combination of light emitting units the synthesized feature amount synthesized from the wavelength feature amounts of the respective wavelengths in the plurality of these light emitting units is, is determined, and a light source including the plurality of light emitting units is configured based on the determined combination of wavelength feature amounts. By so doing, it is possible to separate the diffracted light of each wavelength incident on each pixel based on this unique wavelength feature amount, so that it is possible to easily acquire the PSF of each spatial position and each wavelength from the diffraction image.

Note that, in the present embodiment, even when imaging is executed in a state where different spatial positions are simultaneously turned on, it is possible to easily acquire the PSF of each spatial position and each wavelength in subsequent signal processing. Hereinafter, an example of a condition for making it possible to separate the PSF of each spatial position and each wavelength in the subsequent signal processing, and arrangement of light sources having wavelength features that satisfy this condition will be cited in the following description.

1.2 Mechanism that Diffraction Patterns are Superimposed

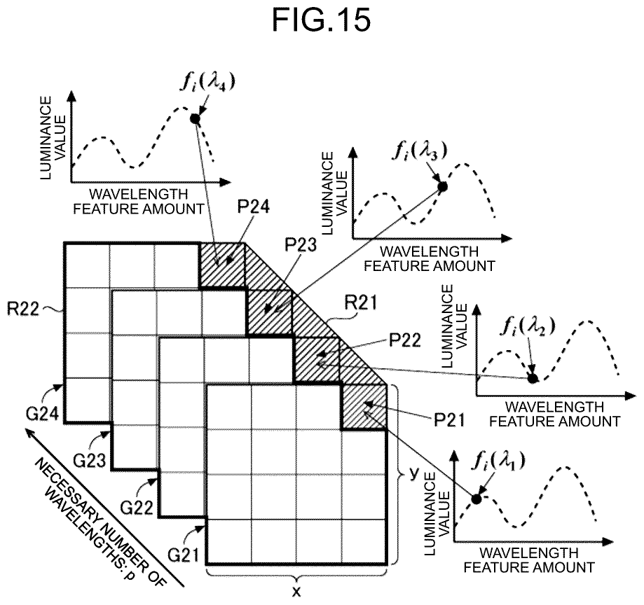

Hereinafter, prior to description of the condition for making it possible to separate the PSF in subsequent signal processing, the mechanism that diffraction patterns of respective wavelengths are superimposed on the diffraction image will be described first in more detail. FIG. 15 is a view for describing the mechanism that diffraction patterns of respective wavelengths are superimposed on a diffraction image. Note that, although the following description will describe a case where light of different wavelengths is radiated from 16 spatial positions of 4×4 in total at which light sources are respectively arranged, deviation of a region at which a diffraction pattern of each wavelength is formed is one pixel unit, and a size of an image at each spatial position formed on a light reception surface (also referred to as an imaging surface) of the light receiving element is equivalent to a size of each pixel in the light receiving element, the present invention is not limited to these conditions. Furthermore, although an example of overlap of diffraction patterns formed in the oblique upper left direction from a 0th-order diffraction pattern on the light reception surface will be cited and described with reference to FIG. 15, the same may be applied to diffraction patterns formed in other directions. Furthermore, this description assumes that the number of types of wavelengths (hereinafter, also referred to as the number of wavelengths) p necessary for generating the PSF is four of the wavelengths λ₁, λ₂, λ₃, and λ₄, a diffraction pattern G21 is a diffraction pattern of light of the wavelength λ₁, a diffraction pattern G22 is a diffraction pattern of light of the wavelength λ₂, a diffraction pattern G23 is a diffraction pattern of light of the wavelength λ₃, and a diffraction pattern G24 is a diffraction pattern of light of the wavelength λ₄.

As illustrated in FIG. 15, a specific region (hereinafter, also referred to as a position of interest) in the diffraction pattern of a certain wavelength in the region in which a diffraction pattern is projected in the diffraction image is not superimposed on a region corresponding to this specific region in the diffraction pattern of another wavelength. More specifically, for example, the position of interest (also referred to as a pixel of interest) P21 in the diffraction pattern G21 associated with the wavelength λ1 is not superimposed on positions of interest P22, P23, and P24 corresponding to the position of interest in the diffraction patterns G22, G32, and G24. The same applies even when the position of interest is any position (e.g., corresponding to a pixel) in the diffraction pattern. Consequently, it is not necessary in the example illustrated in FIG. 15 to consider that diffracted light having the same wavelength feature amount between the pixels corresponding to each other in the diffraction patterns G21, G22, G23, and G34 is incident in a region R22 indicated by diagonal hatching. This means that superimposition in the wavelength direction may be considered for the pixels corresponding to each other in the diffraction patterns G21, G22, G23, and G34 based on feature amounts acquired from pixels other than the pixels corresponding to each other.

Therefore, according to the present embodiment, by using the above mechanism, the condition necessary for making it possible to separate the PSF of each spatial position and each wavelength in the subsequent signal processing is specified.

1.3 Condition for Making it Possible to Separate Wavelength Feature Amount

Next, a method for obtaining a condition for making it possible to separate each wavelength feature amount in subsequent signal processing will be described. In a case where a visual field range necessary for creating the PSF is horizontal X×vertical Y, and the number of necessary wavelengths is p, a size K of a data cube to be created can be expressed by following equation (8).

K=X×Y×p (8)

Furthermore, a sum (corresponding to the synthesized feature amount f*) M of the wavelength feature amounts of the synthesized diffracted light incident on respective pixels in the region R22 in which the wavelength feature amounts are likely to be superimposed is expressed by following equation (9).

M=K−X×Y−p (9)

At this time, the set A of the combination of the wavelength feature amounts of the pixel of interest P21 and the combination of the wavelength feature amounts of the respective pixels in the region R22, more specifically, the set A of the combination of the light emitting units (spatial positions) that radiate the light incident on the pixel of interest P21 and the combination of the light emitting units (spatial positions) that radiate the light incident on the respective pixels in the region R22 is expressed by following equation (10). Note that N represents the number of wavelength feature amounts to be superimposed in equation (10). Therefore, a maximum value of N is the same number of visual field ranges (i.e., X×Y).

A=_{M}C_{N} (10)

Furthermore, the condition for making it possible to separate each wavelength feature amount in subsequent signal processing even in a case where the wavelength feature amounts are superimposed as described above can be described as in following equation (11).

$$∃ {IA}^{′} ; ∀ {A}^{′} ∈ A ° ⋂ ° {\sum }_{i = 1}^{length ( {A}^{′} )} {f}_{{A}^{′ ( i )}}^{′} ( {λ}_{i} ) \qquad ( 11 )$$

That is, the condition for making it possible to separate the wavelength feature amounts in the subsequent signal processing can be that, a total value of the wavelength feature amounts of a combination indicated by an arbitrary element (A′) of the set A is unique (i.e., the total value does not match with the total value of the wavelength feature amounts of other combinations) in the set A, and the correspondence between the observation value (luminance value) observed in each pixel and the combination of the wavelength feature amounts is one to one.

1.4 Method for Determining Wavelength Feature Amount

Next, a method for determining a wavelength feature amount that satisfies the above-described condition will be described. Upon determination of the wavelength feature amount, a subset F of wavelength feature amounts of the data cube D that can be generated from the diffraction image can be described by following equation (12).

F={f₁,f₂, . . . ,f_{X·Y·ρ}} (12)

Hence, the present embodiment proposes an optimization equation expressed by following equation (13) as a conditional equation for determining the subset F to satisfy the condition of above equation (12).

$$∃ F , ( ∀ f ∈ F , {\sum }_{i = 1}^{length ( {A}^{′} )} {f}_{X ( i )} ( {λ}_{i} ) \neq ∀ {f}^{′} ∈ {F}^{′} , {\sum }_{i = 1}^{length ( {B}^{′} )} {f}_{{B}^{′} ( i )}^{′} ( {λ}_{i} ) ) \qquad ( 13 )$$

$$When ( {F}^{′} ⊂ F ⋂ f ∉ {F}^{′} ) ⋂ ( ∀ {A}^{′} ∈ A ⋂ A = {}_{M} {C}_{N} ) ⋂ ( ∀ {B}^{′} ∈ B ⋂ B ⊂ A )$$

1.5 Example of Wavelength Feature Amount Determining Operation

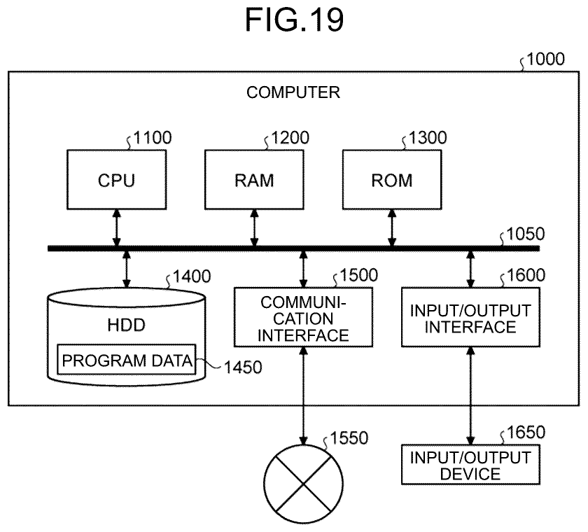

Next, an example of an operation of solving above-described equation (13), that is, an operation of determining the wavelength feature amount that satisfies the above-described condition will be cited and described. Note that the present embodiment will cite an example of an operation of determining the wavelength feature amount that satisfies the condition for making it possible to separate each wavelength feature amount in the subsequent signal processing by solving equation (13) based on random search. Note that the following operation may be executed when, for example, a CPU 1100 (see FIG. 19) in an information processing device 1000 described later executes a predetermined program. Hereinafter, an operation executed by the information processing device 1000 will be described.

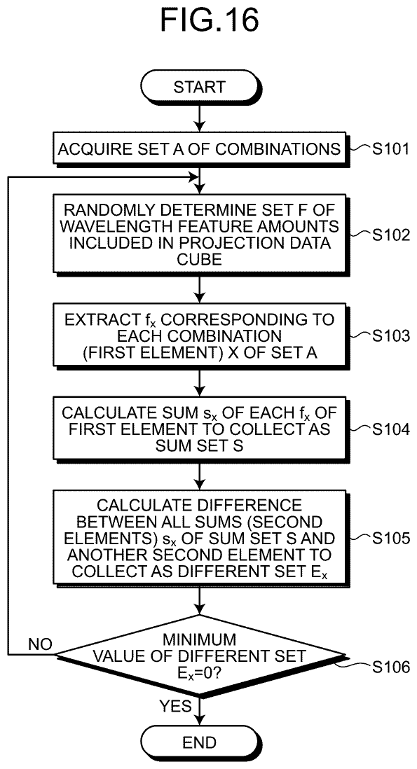

FIG. 16 is a flowchart illustrating an operation example according to the present embodiment. As illustrated in FIG. 16, according to this operation, for each pixel (more specifically, for example, each pixel in a region in which a diffraction pattern is formed) constituting the light receiving element, the information processing device 1000 first acquires the set A of a combination of wavelength feature amounts of this pixel and a combination of wavelength feature amounts of pixels other than this pixel (Step S101). The set A of the combinations can be obtained by calculating, for example, above-described equation (10).

Next, the information processing device 1000 randomly determines the set F (hereinafter, also referred to as a projection data cube) of combinations of wavelength feature amounts of light radiated by each light emitting unit (spatial position) from the light source (Step S102). Note that the set F determined in Step S102 is a candidate of a combination of wavelength feature amounts used as light sources, and is not determined as a combination included in the set F. Furthermore, this subset F may be determined using, for example, a random sequence such as a pseudo random number generated by a random number generator or the like.

Next, the information processing device 1000 extracts the wavelength feature amount fₓ corresponding to each element constituting the set A, that is, each combination (hereinafter, also referred to as a first element) X of the light emitting units (spatial positions) (Step S103). More specifically, the wavelength feature amount fₓ of the light radiated by each light emitting unit (spatial position) to be combined is extracted for each first element.

Subsequently, the information processing device 1000 calculates a sum sₓ of the wavelength feature amounts fₓ for each first element X, and collects the calculated sum sₓ of each first element X as a sum set S (Step S104).

Next, the information processing device 1000 calculates a difference between a sum (hereinafter, also referred to as a second element) sₓ constituting the sum set S and the other second elements, and collects the calculated difference as a difference set Ex (Step S105).

Furthermore, the information processing device 1000 determines whether or not a minimum value of differences (hereinafter, also referred to as third elements) constituting the difference set Ex is zero (Step S106), adopts the projection data cube determined in Step S102 as a light source for calibration in a case where the minimum value is zero (YES in Step S106), and ends this operation. On the other hand, in a case where the minimum value is not zero (NO in Step S106), the information processing device 1000 returns to Step S102, and repeatedly executes the subsequent operations until the minimum value becomes zero.

1.6 Modification of Wavelength Feature Amount Determining Operation

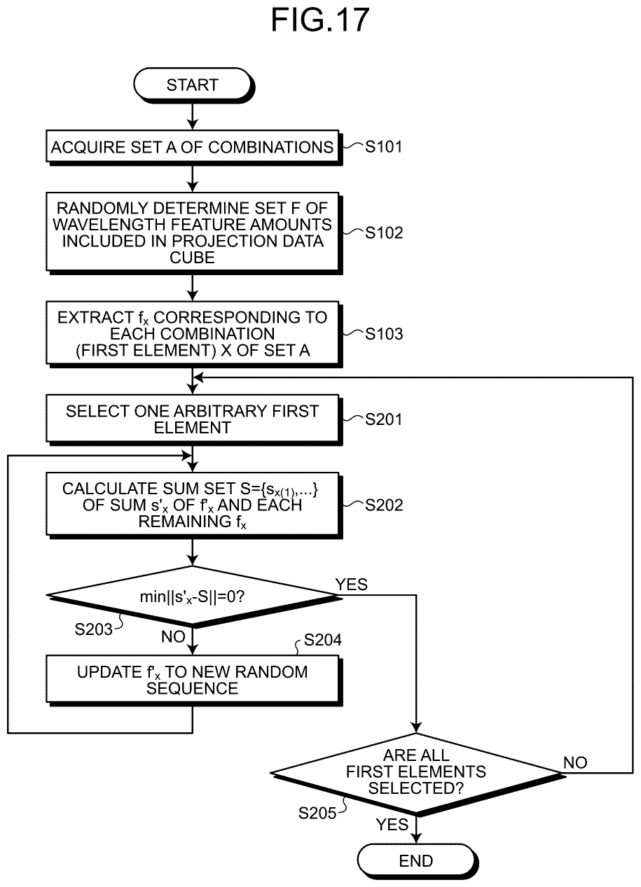

FIG. 17 is a flowchart illustrating an operation example according to the modification of the present embodiment. As illustrated in FIG. 17, according to this operation, first, the information processing device 1000 executes operations similar to Steps S101 to S103 in FIG. 16 to randomly determine a projection data cube, and extract each wavelength feature amount fₓ corresponding to each first element constituting the set A.

Next, the information processing device 1000 selects any one of the first elements X constituting the set A (Step S201).

Next, the information processing device 1000 calculates the sum set S={s_{X(1)}, . . . } of the sum s′_{X} of wavelength feature amounts f′_{X} of the selected first elements X′, and the sum s_{X} of the wavelength feature amounts f_{X} of the other first elements X (Step S202).

Next, the information processing device 1000 determines whether or not a minimum value of the difference between the sum s′_{X} of the selected first elements X′ and the sum s_{X} of the other first element X is zero (Step S203), updates the wavelength feature amount f′_{X} that is being selected using a new random sequence (Step S204) in a case where the minimum value is not zero (NO in Step S203), returns to Step S202, and executes subsequent operations.

On the other hand, in a case where the minimum value is zero (YES in Step S203), the information processing device 1000 determines whether or not all of the first elements X constituting the set A have been selected in Step S201 (Step S205), adopts a projection data cube configured at a current point of time as a light source for calibration in a case where all of the first elements X have been selected (YES in Step S205), and ends this operation. On the other hand, in a case where the first elements X have not been selected (NO in Step S205), the information processing device 1000 returns to Step S201, and repeatedly executes the subsequent operations until all of the first elements X are selected.

1.7 Regarding Restoration of PSF that Uses Reference Table

The correspondence between the wavelength feature amount that is determined by the above-described estimation of an optimum solution (equation (13), FIG. 16, or FIG. 17) and is the condition for making it possible to separate each wavelength feature amount in the subsequent signal processing, and an observation value (luminance value) that can be observed in each pixel may be managed using, for example, a lookup table (hereinafter, also referred to as a reference table).

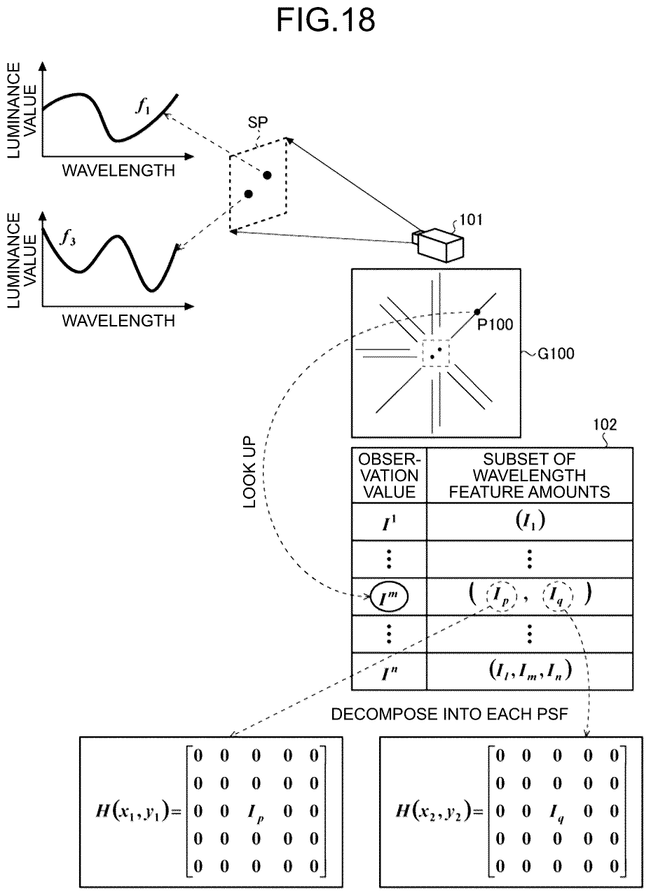

FIG. 18 is a view for describing the PSF restoration operation that uses the reference table according to the present embodiment. Note that a reference table 102 indicating the correspondence relationship between the observation value and the wavelength feature amount is generated by calibration executed before the operation illustrated in FIG. 18, and is appropriately stored in a storage area of a RAM 1200, an HDD 1400, or the like (see FIG. 19) in the information processing device 1000 described later.

As illustrated in FIG. 18, when a plurality of diffraction images G100 are generated by imaging a space SP that emits light of different wavelength feature amounts that change according to predetermined wavelength characteristics (corresponding to, for example, wavelength characteristics f₁ to f₄ illustrated in FIG. 10A) from each spatial position (corresponding to, for example, the spatial positions SP1 to SP16) at a predetermined frame rate using a spectral camera 101 during observation after calibration, for example, the information processing device 1000 specifies the subset F of combinations of wavelength feature amounts for respective pixels by looking up in the reference table 102 using the observation value (luminance value) of each pixel for each diffraction image G100. Furthermore, the information processing device 1000 generates the PSF of each wavelength of diffracted light incident on each pixel by using the wavelength feature amount included in the specified subset F.

This will be more specifically described focusing on a pixel P100 in the diffraction image G100. Note that FIG. 18 assumes that the observation value (luminance value) of the pixel P100 in the diffraction image G100 includes the wavelength feature amounts of the light of the wavelength characteristics f₁ and f₃ respectively emitted from the two spatial positions in the space SP. When the diffraction image G100 is acquired using the spectral camera 101, the information processing device 1000 specifies a subset (Iₚ, I_{q}) of the wavelength feature amounts of the light incident on the pixel P100 by looking up in the reference table 102 using a luminance value I^{m} of the pixel P100. Furthermore, the information processing device 1000 generates a PSF (H (x1, y1), H (x2, y2)) of each of the light of the wavelength λ₁ and the light of the wavelength λ₃ incident on the pixel P100 by using the wavelength feature amounts Iₚ and I_{q} included in the specified subset.

By executing the above operation on all pixels constituting the diffraction image G100, it is possible to restore a normal image, that is, an image that does not include the diffraction pattern, from the diffraction image G100 acquired by the spectral camera 101.

1.8 Conclusion

As described above, according to the present embodiment, it is possible to acquire the PSF of each spatial position and each wavelength by performing photographing once, so that it is possible to reduce cost required for calibration, and more easily acquire the PSF.

### 2. Hardware Configuration

Various processing according to the above-described embodiment can be realized by the information processing device 1000 employing, for example, a configuration as illustrated in FIG. 19. That is, the information processing device 1000 can function as a determination unit and a generation unit in the claims. FIG. 19 is a hardware configuration diagram illustrating an example of the information processing device 1000 that executes various processing according to the present disclosure. The information processing device 1000 includes the CPU 1100, the RAM 1200, a Read Only Memory (ROM) 1300, the Hard Disk Drive (HDD) 1400, a communication interface 1500, and an input/output interface 1600. Each unit of the information processing device 1000 is connected by a bus 1050.

The CPU 1100 operates based on a program stored in the ROM 1300 or the HDD 1400, and controls each unit. For example, the CPU 1100 develops in the RAM 1200 a program stored in the ROM 1300 or the HDD 1400, and executes processing corresponding to various programs.

The ROM 1300 stores a boot program such as a Basic Input Output System (BIOS) executed by the CPU 1100 when the information processing device 1000 is activated, a program that depends on hardware of the information processing device 1000, and the like.

The HDD 1400 is a recording medium that non-transiently records a program to be executed by the CPU 1100, data used by this program, and the like, and can be read by the information processing device. Specifically, the HDD 1400 is a recording medium that records a program for executing each operation according to the present disclosure that is an example of the program data 1450.

The communication interface 1500 is an interface for the information processing device 1000 to connect with an external network 1550 (e.g., the Internet). For example, the CPU 1100 receives data from another equipment, and transmits data generated by the CPU 1100 to the another equipment via the communication interface 1500.

The input/output interface 1600 employs a configuration including the above-described I/F unit 18, and is an interface for connecting an input/output device 1650 and the information processing device 1000. For example, the CPU 1100 receives data from an input device such as a keyboard and a mouse via the input/output interface 1600. Furthermore, the CPU 1100 transmits data to an output device such as a display, a speaker, or a printer via the input/output interface 1600. Furthermore, the input/output interface 1600 may function as a media interface that reads a program or the like recorded on predetermined recording media (media). The media are, for example, optical recording media such as a Digital Versatile Disc (DVD) and a Phase change rewritable Disk (PD), magneto-optical recording media such as a Magneto-Optical disk (MO), tape media, magnetic recording media, semiconductor memories, or the like.

In a case where, for example, the information processing device 1000 executes various processing according to the above-described embodiment, the CPU 1100 of the information processing device 1000 executes various processing by executing a program loaded on the RAM 1200. Furthermore, the HDD 1400 stores programs and the like according to the present disclosure. Note that, although the CPU 1100 reads the program data 1450 from the HDD 1400 to execute, these programs may be acquired from another device via the external network 1550 in another example.

Although the embodiment of the present disclosure has been described above, the technical scope of the present disclosure is not limited to the above-described embodiment as is, and various modifications can be made without departing from the gist of the present disclosure. Furthermore, components according to different embodiments and modifications may be appropriately combined.

Furthermore, the effects according to the embodiment described in the description are merely examples and are not limited thereto, and other effects may be provided.

Furthermore, the above-described embodiment may be used alone, or may be combined with another embodiment and used.

Note that the present technique can also employ the following configurations.
(1)

An information processing device including:
a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other;
a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations; and
a generation unit that generates a point spread function of each wavelength based on the third combination of each pixel specified by the specifying unit.
(2)

The information processing device according to (1), wherein
the first combinations are combinations of wavelength feature amounts of light that can be incident on an identical pixel specified based on overlap of diffraction patterns that can be formed by causing the light from the light source to be incident on the diffraction element.
(3)

The information processing device according to (1) or (2), wherein
the determination unit determines the two or more second combinations whose total value of the values are unique to each other using a random sequence.
(4)

The information processing device according to any one of (1) to (3), wherein
the determination unit calculates a sum of the wavelength feature amounts of each of the first combinations, and determines a combination whose difference between the calculated sums is zero as the second combination.
(5)

The information processing device according to any one of (1) to (4), wherein
the determination unit selects one first combination from a set of the first combinations, calculates a first sum of wavelength feature amounts of the selected first combination and a second sum of wavelength feature amounts of each of other first combinations in the set, and, when a minimum value of a difference between the first sum and the second sums is zero, determines the selected first combination as the second combination.
(6)

The information processing device according to (5), wherein
the determination unit selects another one first combination from the set when the minimum value of the difference between the first sum and the second sums is not zero.
(7)

The information processing device according to any one of (1) to (6), wherein
the wavelength feature amount is a light intensity of each wavelength.
(8)

An information processing device including:
an imaging unit that images a predetermined space via a diffraction element including a grating pattern; and
a specifying unit that specifies a combination of light incident on each pixel from a luminance value of each pixel in a diffraction image acquired by the imaging unit by using the point spread function generated by the information processing device according to any one of (1) to (7).
(9)

The information processing device according to (8), wherein,
by using a reference table that manages a correspondence between an observation value acquired from each pixel of the imaging unit and the point spread function for separating the observation value into a wavelength feature amount of each wavelength, the specifying unit specifies a combination of the light incident on each pixel from a luminance value of each pixel in the diffraction image acquired by the imaging unit.
(10)

An information processing method including:
determining two or more second combinations among first combinations, the first combinations including one or more wavelength feature amounts selected from a population of two or more wavelength feature amounts having the mutually unique values, and the two or more second combinations including wavelength feature amounts whose total value of values is unique to each other;
imaging, via a diffraction element including a grating pattern, light radiated from a light source and including light corresponding to each of two or more of the wavelength feature amounts determined based on the two or more second combinations;
specifying a third combination of the wavelength feature amounts of the light incident on each pixel from a luminance value of each pixel in a diffraction image acquired by the imaging; and
generating a point spread function of each wavelength based on the third combination of each specified pixel.
(11)

A program for causing a computer to function,
the program causing
the computer to function as:
a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other;
a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations determined by the determination unit; and
a generation unit that generates a point spread function of each wavelength based on the third combination of each pixel specified by the specifying unit.

### REFERENCE SIGNS LIST

101 SPECTRAL CAMERA
102 REFERENCE TABLE
941 OBJECTIVE LENS
942 SLIT
943 COLLIMATING LENS
944 DIFFRACTION GRATING SPECTRAL ELEMENT
945 IMAGING LENS
946 AREA SENSOR
1000 INFORMATION PROCESSING DEVICE
1100 CPU
1200 RAM
1300 ROM
1400 HDD
1500 COMMUNICATION INTERFACE
1550 EXTERNAL NETWORK
1600 INPUT/OUTPUT INTERFACE
1650 INPUT/OUTPUT DEVICE
SP SPACE
SP1 to SP16 SPATIAL POSITION

1. An information processing device including:
a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other;
a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations; and
a generation unit that generates a point spread function of each wavelength based on the third combination of each pixel specified by the specifying unit.

2. The information processing device according to claim 1, wherein
the first combinations are combinations of wavelength feature amounts of light that can be incident on an identical pixel specified based on overlap of diffraction patterns that can be formed by causing the light from the light source to be incident on the diffraction element.

3. The information processing device according to claim 1, wherein
the determination unit determines the two or more second combinations whose total value of the values are unique to each other using a random sequence.

4. The information processing device according to claim 1, wherein
the determination unit calculates a sum of the wavelength feature amounts of each of the first combinations, and determines a combination whose difference between the calculated sums is zero as the second combination.

5. The information processing device according to claim 1, wherein
the determination unit selects one first combination from a set of the first combinations, calculates a first sum of wavelength feature amounts of the selected first combination and a second sum of wavelength feature amounts of each of other first combinations in the set, and, when a minimum value of a difference between the first sum and the second sums is zero, determines the selected first combination as the second combination.

6. The information processing device according to claim 5, wherein
the determination unit selects another one first combination from the set when the minimum value of the difference between the first sum and the second sums is not zero.

7. The information processing device according to claim 1, wherein
the wavelength feature amount is a light intensity of each wavelength.

8. An information processing device including:
an imaging unit that images a predetermined space via a diffraction element including a grating pattern; and
a specifying unit that specifies a combination of light incident on each pixel from a luminance value of each pixel in a diffraction image acquired by the imaging unit by using the point spread function generated by the information processing device according to claim 1.

9. The information processing device according to claim 8, wherein,
by using a reference table that manages a correspondence between an observation value acquired from each pixel of the imaging unit and the point spread function for separating the observation value into a wavelength feature amount of each wavelength, the specifying unit specifies a combination of the light incident on each pixel from a luminance value of each pixel in the diffraction image acquired by the imaging unit.

10. An information processing method including:
determining two or more second combinations among first combinations, the first combinations including one or more wavelength feature amounts selected from a population of two or more wavelength feature amounts having the mutually unique values, and the two or more second combinations including wavelength feature amounts whose total value of values is unique to each other;
imaging, via a diffraction element including a grating pattern, light radiated from a light source and including light corresponding to each of two or more of the wavelength feature amounts determined based on the two or more second combinations;
specifying a third combination of the wavelength feature amounts of the light incident on each pixel from a luminance value of each pixel in a diffraction image acquired by the imaging; and
generating a point spread function of each wavelength based on the third combination of each specified pixel.

11. A program for causing a computer to function,
the program causing
the computer to function as:
a determination unit that determines two or more second combinations among first combinations, the first combinations including one or more wavelength feature amounts selected from a population of two or more wavelength feature amounts having mutually unique values, and the two or more second combinations including wavelength feature amounts whose total value of the values is unique to each other;
a specifying unit that specifies a third combination of the wavelength feature amounts of light incident on each pixel from a luminance value of each pixel in a diffraction image acquired via a diffraction element including a grating pattern by receiving light radiated from a light source and including light corresponding to each of two or more of the wavelength feature amounts determined based on the two or more second combinations determined by the determination unit; and
a generation unit that generates a point spread function of each wavelength based on the third combination of each pixel specified by the specifying unit.
